(19) United States
(12) Patent Application Publication
(10) Pub. No.: US 2009/0318909 A1
(43) Pub. Date: Dec. 24, 2009

(54) APPARATUS AND METHOD FOR ABLATION-RELATED DERMATOLOGICAL TREATMENT OF SELECTED TARGETS

(75) Inventors: Leonard C. DeBenedictis, Palo Alto, CA (US); George Frangineas, Fremont, CA (US)

Correspondence Address: WOOD , HERRON & EVANS, LLP (SOLTA), 441 VINE STREET, 2700 CAREW TOWER, CINCINNATI, OH, 45202, US

(73) Assignee: SOLTA MEDICAL, INC., Hayward, CA (US)

(21) Appl. No.: 12/497,487

(22) Filed: Jul. 2, 2009

Related U.S. Application Data
(63) Continuation of application No. 11/747,663, filed on May 11, 2007, now abandoned.
(60) Provisional application No. 60/800,075, filed on May 11, 2006.

(51) Int. Cl. A61B 18/20; A61B 18/18

(52) U.S. Cl. 606/9; 606/33

(57) ABSTRACT

The invention describes a treatment for skin containing selected targets that provides feedback in response to a measurement enabled by the ablation of holes. The inventive apparatus includes an electromagnetic source configured to emit ablative electromagnetic energy, a delivery system, a sensing element, and a controller. The delivery system can be configured to receive ablative energy from the electromagnetic source and deliver it to multiple discrete locations at the selected region to form a pattern of discrete holes in epidermal and dermal tissue of the skin. The lipid content a portion of the tissue can be evaluated using a sensing element. At least one pulse of electromagnetic energy is delivered to the skin under control of a controller in response to the result of a measurement by the sensing element. The apparatus may include a positional sensor to provide additional dosage control, particularly when the inventive method is used with a continuously movable handpiece.

### CROSS-REFERENCE TO RELATED APPLICATION(S)

This application claims priority under 35 U.S.C. § 119(e) to U.S. Provisional Patent Application Ser. No. 60/800,075, “Apparatus and Method for Ablation-Related Dermatological Treatment of Selected Targets,” filed May 11, 2006, which is incorporated by reference herein in its entirety.

### BACKGROUND OF THE INVENTION

1. Field of the Invention

This invention relates generally to actively controlled dermatological treatment of skin. More particularly, it relates to a method and apparatus for dermatological treatment that use an electromagnetic source to ablate holes in the skin and a feedback system to control the treatment in connection with the ablation.

2. Description of the Related Art

Lipid-rich tissues and regions are common targets for dermatological treatments. Examples of lipid-rich targets are sebaceous glands, sebaceous cysts, and subcutaneous fat. Broad area treatments require a large amount of energy to treat lipid-rich targets which are typically large and located at least 1 millimeter (mm) deep in tissue. The large amount of energy required for effective treatment causes side effects. A number of inventors such as Tankovich et al. and Altshuler et al. have developed approaches to treat lipid-rich targets.

For example, U.S. Pat. No. 5,817,089 by Tankovich et al. describes the use of absorbing particles that are deposited on the surface of the skin and penetrate into the sebaceous glands where they are exploded using selective photothermolysis. This approach requires messy carbon particles to be deposited on the skin, has limited efficacy due to limited penetration of particles into the desired treatment areas, and only addresses targets that are open at the surface to allow penetration by the absorbing particles. Plugged targets, such as clogged pores, may not be treated because the absorbing particles cannot penetrate beyond the clogged opening.

U.S. Pat. No. 6,605,080 by Altshuler et al describes a different approach for treating lipid-rich targets. Treatment is performed with wavelengths that are more strongly absorbed by human fatty tissue than in water. The chosen wavelengths can be used to provide selective absorption in lipid-rich targets in comparison to surrounding tissue that is comprised of mainly water. Appropriate wavelengths can be determined from FIGS. 1 and 2, which are copied from Altshuler et al. Even using the selected wavelengths, overtreatment and undertreatment are problems due to the lack of feedback and spatial selectivity with the delivered energy.

U.S. Pat. No. 6,997,923 by Anderson et al and copending U.S. patent application No. 60/773,192 by DeBenedictis et al. describe apparatuses and methods that promote rapid healing of targets by sparing healthy skin surrounding treatment zones. DeBenedictis et al. further describes the drilling of holes in skin. However, both of these can be improved by better active targeting of lipid-rich targets and/or by better use of feedback mechanisms. Such active targeting and feedback can allow additional sparing of tissue that allows for fewer side effects and thus can permit more effective treatment at higher treatment levels.

Thus, there is a need for a method and apparatus that better controls delivery of treatment energy by providing feedback in response to measurements, for example as enabled by the ablation of holes and/or in response to the measured lipid content of the target tissue.

### SUMMARY OF THE INVENTION

The present invention overcomes the limitations of the prior art and improves the treatment of selected targets in skin by providing feedback in response to measurement enabled by the ablation of holes and/or in response to the measured lipid content of the target tissue. Examples of selected targets are lipid-rich targets, foreign bodies (e.g. tattoo ink, cancers, and PDT drugs), hair follicles, hair bulge cells, and vascular tissue.

In one aspect of the inventive method, holes are ablated in epidermal and dermal tissue of the skin. A sensing element is used to evaluate at least a portion of the tissue that is somehow affected by the ablation. For example, the property of the tissue may change as a function of ablation. Alternately, the ablation may enable access to tissue or measurements that were previously not accessible. A controller controls the delivery of a controlled pulse to the selected region based on feedback from the sensing element.

The evaluation step may comprise the measurement of at least one characteristic of a portion of the ablated tissue. For example, the ablation rate, optical scattering properties, optical absorption properties, fluorescent emission properties, or a combination thereof can be measured. Multiple illumination or detection wavelengths can be used to improve the sensitivity and selectivity of optical measurements.

The evaluation step may comprise the measurement of at least one characteristic of the remaining tissue, where the characteristic or access to the tissue is affected by the ablation. For example, an acoustical or radio-frequency absorption spectrum that is affected by the ablation process can be measured. In another embodiment, the depth of at least one hole is measured. In yet another embodiment, the measurement of the remaining tissue involves the measurement of a scattering property, an absorption property, fluorescent emission, or a combination thereof using at least one optical wavelength, for example where these properties are affected by the ablation or the ablation enables access to the tissue. To improve the sensitivity and selectivity of optical measurements, multiple wavelengths can be detected or used for illumination.

The lipid content of the ablated or remaining tissue may be measured during the evaluation step.

The evaluation step can use a sensing element to measure a signal that is generated as a result of the ablating step. For example, an acoustic transducer or imaging system can be used to capture an acoustic signal generated as the result of ablation or an image of an ablation event.

In response to the evaluating step, a controller can be used to control the delivery of subsequent treatment energy to the target area. In some embodiments, the controller controls the energy delivery rate and/or the wavelength of the electromagnetic source. The electromagnetic source can be a laser. The energy delivery rate of the electromagnetic source may be controlled, for example, by changing the power level, the pulse repetition frequency, the pulse duty cycle, or a combination thereof. In some embodiments, the electromagnetic source is a laser and the energy delivery rate and/or the wavelength of the laser is reduced in response to the detection of a lipid-rich target during the evaluation step. In some embodiments, the controlling step is the activating of the electromagnetic source to generate the controlled pulse.

In some embodiments, the controlled pulse is delivered into one or more holes created during the ablation step. In some embodiments, the majority of the optical energy in the controlled pulse does not extend beyond the edge of the holes created during the ablation step.

The electromagnetic source can be an optical, radio frequency (RF), or RF plasma source. The electromagnetic source may comprise multiple sources or may comprise only a single source. In some embodiments, the electromagnetic source comprises an ablative source and a source that is nonablative. In some embodiments, the electromagnetic source may comprise a laser, an optical amplifier, a fiber laser, a fiber amplifier, or a combination thereof. The optical source may further comprise a Raman-shifting element to shift the wavelength of the emitted electromagnetic energy to a desired wavelength. In some embodiments, the electromagnetic source comprises an optical source that emits a nonnegligible amount of energy at a fat selective wavelength.

In some embodiments, the ablating step is performed by directing one or more pulses from a laser to the selected region.

The electromagnetic source can be an ablative or a nonablative laser. Examples of ablative lasers that could be used are a CO₂ laser, a thulium-doped fiber laser, an Er:YAG laser, and a holmium laser. Another example of an ablative laser that could be used is a thulium-doped fiber laser that is tunable (either discretely tunable, continuously tunable, or some combination thereof). The beam from the ablative laser can be directed to the selected region of skin to heat water in the tissue to cause ablation. The ablative laser can be used to create at least two discrete holes in a pattern corresponding to the optical intensity profile of the beam.

In embodiments where the electromagnetic source comprises an ablative laser, the controlled pulse may be emitted by the ablative laser or by a second source, for example a second laser. Either the ablative laser or the second laser can be used to cause treatment of a lipid-rich target.

In embodiments in which the electromagnetic source comprises an ablative laser, the electromagnetic source can comprise a second source that produces a controlled pulse with a different electromagnetic spectrum than the ablative laser. For example, the ablative laser may be a CO₂ laser and the second source may be a Raman-shifted fiber laser, an erbium-doped fiber laser, a seeded erbium-doped fiber amplifier, a flashlamp, an RF source, or a combination thereof.

In some embodiments, the holes are ablated with a laser having a water absorbed wavelength and the controlled pulse is produced by a laser emitting a fat selective wavelength.

In some embodiments, the holes are ablated with a laser having a water absorbed wavelength and the controlled pulse is produced by a laser emitting a water absorbed wavelength.

In some embodiments, an absorbing agent may be applied to the surface of the selected region and the ablating step comprises the step of directing a laser to the absorbing agent.

The density of holes created during treatment in the selected region is preferably 100-10,000 holes per square centimeter, and more preferably 1000-2000 holes per square centimeter. Each hole preferably has a depth of 0.5-6.0 mm and more preferably from 1-2 mm. Each hole preferably has a diameter of 0.2-2.0 mm and more preferably from 0.3-1.0 mm. All combinations of each of these hole depth and diameter ranges are within the scope of the invention.

In some embodiments, the controlled pulse can be delivered using an optical scanner, an optical lens array, a patterned mask, or a cooled patterned mask. A scanner could be used to direct the controlled pulse to a location within the selected reigon.

The surface of the selected region may be cooled in some embodiments to spare the epidermis or reduce side effects.

Certain aspects of the inventive method may further comprise the step of measuring a positional parameter of the handpiece. Examples of handpiece positional parameters are speed, velocity, acceleration, or position relative to the selected area. The positional parameters can be measured with a positional sensor. Examples of positional sensors are an optical mouse chip, a mechanical mouse, a CCD, a capacitive array sensor, an accelerometer, and a gyroscope.

Other aspects of the invention include apparatus designed to accomplish the aforementioned inventive methods. The inventive apparatus can include an electromagnetic source configured to emit ablative electromagnetic energy, a delivery system, a sensing element, and a controller. The delivery system can be configured to receive ablative energy from the electromagnetic source and deliver it to multiple discrete locations at the selected region to form a pattern of discrete holes in the skin, preferably of the size and with the areal density described above.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 (prior art) is a graph describing the optical absorption spectra of human fatty tissue and water.

FIG. 2 (prior art) is a graph describing the ratio of optical absorption coefficients of human fatty tissue and water as a function of wavelength.

FIG. 3 is a diagram showing an embodiment of the invention.

FIGS. 4A-4D are illustrations of the skin. FIG. 4A shows untreated skin with two lipid-rich targets. FIGS. 4B-4D show illustrative examples of the skin following treatment according to embodiments of the inventive apparatus and method.

FIGS. 5 and 6 are diagrams of additional embodiments of the invention.

FIG. 7 is a flow chart describing an embodiment of the inventive method.

### DETAILED DESCRIPTION OF THE PREFERRED EMBODIMENTS

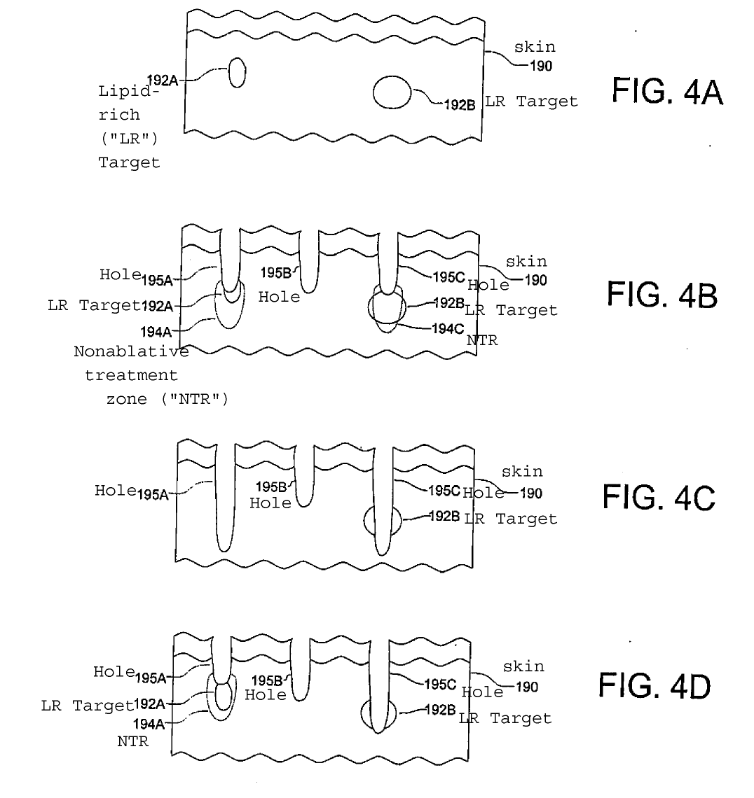

The example inventive system illustrated in FIG. 3 includes a controller 150 that controls an electromagnetic source 110 that emits one or more pulses of electromagnetic energy 115. A delivery system 140 is configured to receive and direct the electromagnetic energy 115 from the electromagnetic source 110 to a target region of skin 190 to create holes 195 in the skin 190. The system further comprises a positional sensor 160 and a sensing element 170 that each provide feedback to the controller 150. The electromagnetic energy 115 that is delivered to the skin 190 can be adjusted or triggered by the controller 150 in response to signals received from the positional sensor 160, the sensing element 170, or a combination thereof. The controller 150 can control the treatment by adjusting parameters of the electromagnetic source 110, the delivery system 140, or a combination thereof. One or more components of the system may be contained in a handpiece 100 that allows manual control over delivery of the electromagnetic energy 115 to the skin 190. In the embodiment pictured in FIG. 3, the handpiece contains the delivery system 140, the sensing element 170, and the positional sensor 160.

In this example, the electromagnetic source 110 is used to create both the ablation and the controlled pulse. In this application, the term “controlled pulse” means one or more pulses of electromagnetic energy 115 emitted by the electromagnetic source 110. The controlled pulse is controlled by the controller 150 in response to a signal from the sensing element 170.

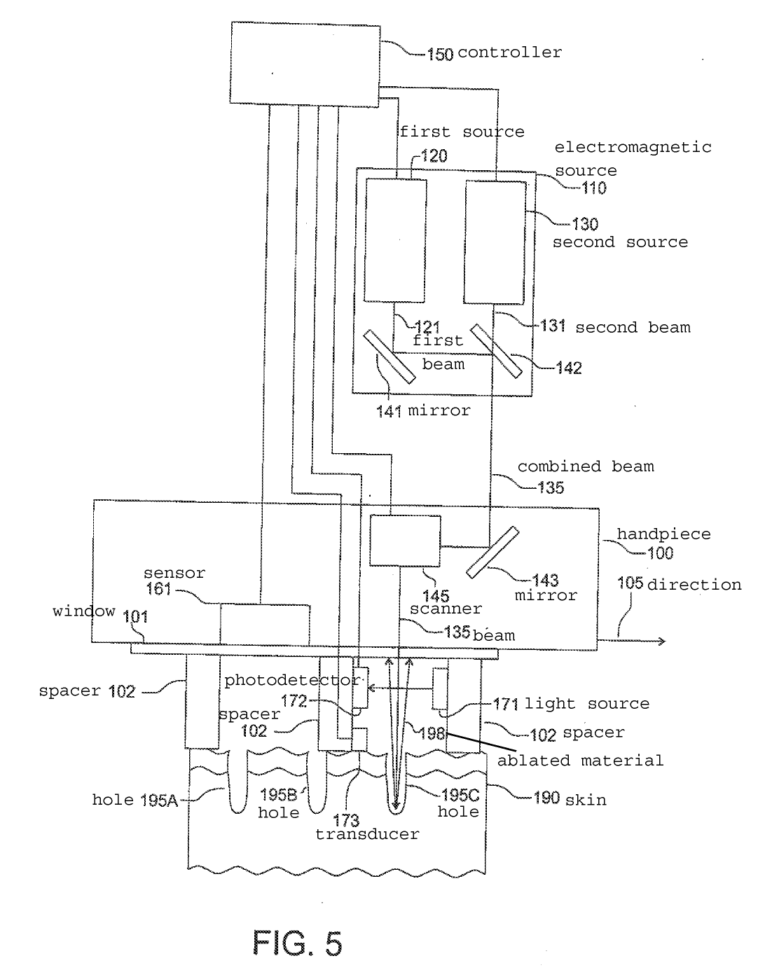

Through the choice of sensing element 170, electromagnetic source 110, and software implementation in the controller 150, the apparatus of FIG. 3 can be used to create different types of desired treatment responses. Examples of how the inventive system can be used are shown in FIGS. 4A-4D. The skin 190 shown in FIG. 4A contains two lipid-rich targets 192A,B and can be treated by the inventive apparatus to create the desirable outcomes shown in FIGS. 4B-4D.

In FIGS. 4B and 4C, holes are drilled using a predefined set of ablation parameters. This can create a series of holes that are approximately uniform in depth. If, during the ablation step, a lipid-rich target is detected by the sensing element 170, either in the ablated tissue or in the region underneath the hole, then the electromagnetic source 110 or the delivery system 140 can be directed by the controller to deliver nonablative thermal treatment energy to create nonablative treatment zones 194A,C, as illustrated in FIG. 4B. Alternately, the electromagnetic source 110 or the delivery system 140 can be directed by the controller to continue to deliver ablative energy to drill the holes 195A,C deeper into the skin 190, perhaps using a second set of predetermined parameters, as illustrated in FIG. 4C. For example, the differences between the first (ablative) and second parameter sets could comprise one or more of wavelength, pulse energy, surface cooling, spot size, focal depth, and energy delivery rate of the electromagnetic energy 115.

In yet another preferred embodiment, the controller 150 can direct the electromagnetic source 110 or the delivery system 140 to alter treatment as soon as a lipid-rich target is detected by the sensing element 170. In the example illustrated by FIG. 4D, a first hole 195A is created through ablation until a lipid-rich target 192A is detected. At that time, the controller 150 changes the operating parameters for the electromagnetic source 110 to cause the electromagnetic source 110 to emit nonablative energy to cause thermal treatment of zone 194A. A second hole 195B is created through ablation according to a predefined set of ablation parameters and since no lipid-rich target is discovered during the ablation step for the second hole 195B, the controller 150 does not alter the parameters. A third hole 195C is created through ablation. As the third hole 195C is being ablated, a second lipid-rich target 192B is detected by the sensing element 170. In this example, the controller 150 may evaluate the depth of lipid-rich target 192B within the skin 190 and direct the electromagnetic source 110 to continue to deliver ablative treatment energy until the lipid-rich target 192B is no longer detected in the ablation material or in the region below the third hole 195C.

The holes 195 may be created using an apparatus that incorporates an ablative CO₂ laser as described in U.S. provisional patent application No. 60/773,192 (entitled “Laser system for treatment of skin laxity,” filed Feb. 13, 2006) and in U.S. utility patent application Ser. No. 11/674,654 (entitled “Laser system for treatment of skin laxity,” filed Feb. 13, 2007), which are herein incorporated by reference. For example, each hole may be ablated using a wavelength of approximately 10.6 μm emitted from a CO₂ laser with a pulse energy of 8-20 mJ, a beam diameter at the skin surface of 100-200 μm, and an optical power of 50 W. Nonablative treatment parameters for the second laser can be, for example, a wavelength of 1.55 μm emitted from an erbium-doped fiber laser with a pulse energy of 10-100 mJ, a beam diameter of 80-200 μm and an optical power of 20-30 W.

A source can be both ablative and nonablative depending on the selected parameters and the targeted material. The use of the terms ablative and nonablative refers to the interaction between the source, the chosen parameters, and the target material.

Other variations in timing of response and of combinations of response are considered to be within the scope of the invention. Parameters other than the depth of a lipid-rich target may be used to provide feedback to the system to control treatment. Multiple ablated regions may be treated by a beam that covers multiple holes (not pictured). In some embodiments, the controlled pulse from the electromagnetic source 110 may be beneficially delivered into one or more individual holes so that the majority of the energy in the controlled pulse does not extend beyond the perimeters of one or more of the holes.

Additional embodiments can be described through reference to the elements of FIG. 3 as discussed below.

The positional sensor 160 is an optional component that measures a positional parameter of the handpiece. For example, the positional sensor 160 can measure at least one of a position, velocity, speed, orientation, or acceleration of some part of the handpiece 100 relative to the skin 190. The relative measurements can be used to control the rate of energy delivery or other treatment parameters.

The positional sensor 160 is particularly useful in handpieces that are designed to be moved in a continuous motion, rather than discretely stamped, because the positional sensor 160 can provide feedback to compensate for changes in velocity of the handpiece as the handpiece is moved across the selected treatment area. In a preferred embodiment, the velocity of the handpiece is measured and the power level of the electromagnetic energy 115 is altered to maintain uniform treatment fluence across a selected treatment region. In another preferred embodiment, the pulse repetition rate is altered in response to the speed of the handpiece 100 along a particular direction 105 to deliver an approximately uniform density of treatment zones regardless of relative handpiece speed.

The positional sensor 160 can be an optical mouse chip (e.g., model ADNS-3080 by Avago Technologies, Inc. Palo Alto, Calif.), a mechanical mouse, a capacitive array sensor, an accelerometer, a gyroscope, or other device that senses a relative positional parameter of the handpiece 100. In embodiments wherein the positional sensor 160 is an optical mouse, blue FD&C #1 coloring in water with a concentration of approximately 0.4% by mass can be rubbed onto the skin to improve the responsivity of the positional sensor. Additional examples of suitable positional sensors are described in pending U.S. patent applications Nos. Ser. 11/020,648 (entitled “Method and apparatus for monitoring and controlling laser-induced tissue treatment,” filed Dec. 23, 2004) and 60/712,358 (entitled “Method and apparatus for monitoring and controlling thermally induced tissue treatment,” filed Aug. 29, 2005), which are herein incorporated by reference.

The controller 150 can be a computer or electronics that are designed to control the electromagnetic source 150. As desired, the controller 150 may additionally control the delivery system 140 and may collect data from the positional sensor 160, the sensing element 170, or a combination thereof.

The delivery system 140 is chosen based on the type of electromagnetic source 110 that is selected. For example, if the electromagnetic source 110 comprises an RF source, then the delivery system 140 could include wires, a phased array antenna, waveguide, and contact pads to deliver RF treatment energy to the skin 190. In embodiments wherein the electromagnetic source 110 comprises an optical source, then the delivery system 140 could be an optical scanner, an optical fiber, a patterned mask, mirrors, lenses, a lens array, or a combination thereof Examples of suitable optical scanners are galvanometer based scanners (Cambridge Technology, Inc., Cambridge, Mass.), polygon scanners, MEMS scanners, counter-rotating scanners and starburst scanners. Examples of suitable counter-rotating and starburst scanners are described, respectively, in more detail in copending U.S. patent applications Ser. No. 10/750,790 (entitled “High speed, high efficiency optical pattern generator using rotating optical elements,” filed Dec. 31, 2003) and 11/158,907 (entitled “Optical pattern generator using a single rotating component,” filed Jun. 20, 2005), both of which are herein incorporated by reference. A scanning delivery system 140 can be synchronized with the triggering of the electromagnetic source 110 by the controller 150, which can additionally use feedback from the positional sensor 160 to control the rate of treatment to deliver a desired treatment density.

The sensing element 170 detects one or more parameters that result, at least in part, from the ablation of one or more holes in the skin 190. The sensing element 170 can, for example, detect one or more of the following parameters: the depth of one or more holes, the lipid content of the ablated material, the ablation rate of the ablated material, and the acoustic signal generated during ablation. The sensing element can sense a characteristic of the ablated material or a characteristic of the remaining tissue (i.e. tissue that has not yet been ablated, for example the tissue underlying at least one of the holes and exposed by the ablation).

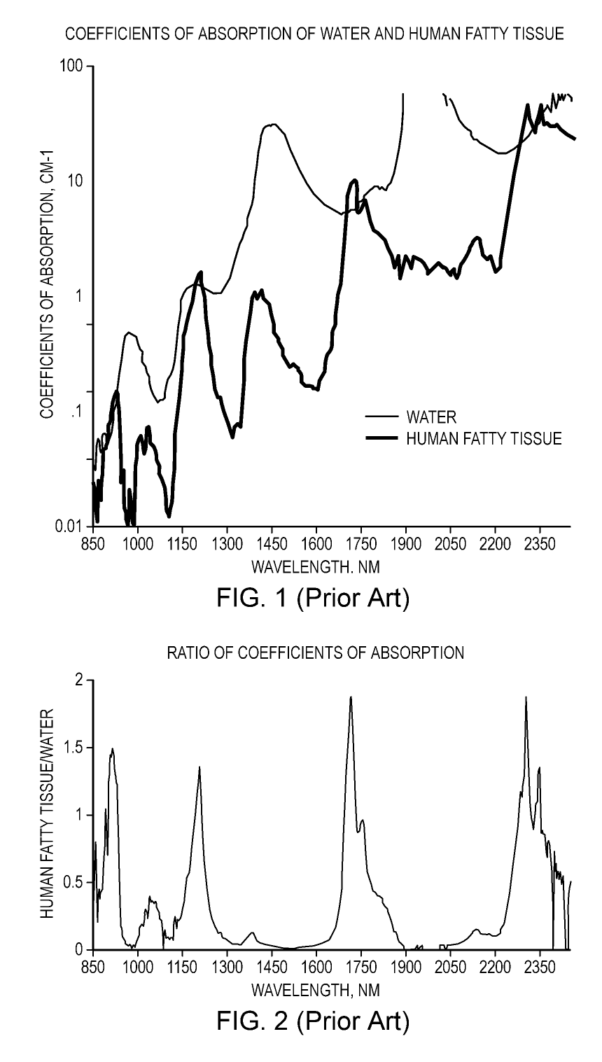

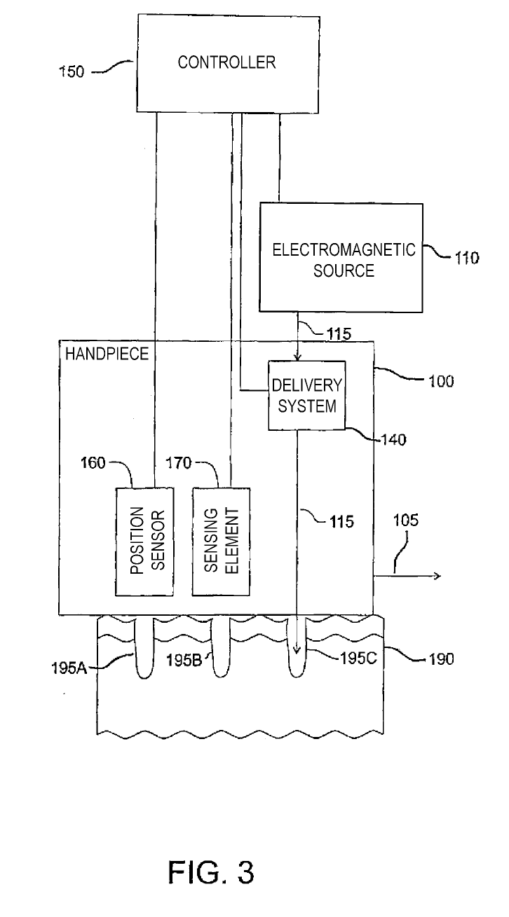

The sensing element 170 can be a spectral sensor that measures the spectral absorption or scattering characteristics of tissue ablated from the hole or of tissue at the base of the hole. The spectral characteristics of ablated tissue may be measured as the tissue is ablated from the skin 190 or after it comes to rest on a debris collection plate. One example of a spectral sensor is a broad band illumination source, a linear photodetector array, and a diffraction grating that spreads the spectral signal penetrating through the ablated material. Other suitable spectral sensors for measuring absorption, scattering, or a combination thereof for two or more wavelengths are well known in the art. Using multiple wavelengths will provide a better signal to detect the presence of a particular lipid target than would using a single wavelength. Spectral sensors are particularly useful for distinguishing particular types of targets according to a spectral signature. Examples of selected targets that can be targeted are lipid-rich tissue, foreign bodies (e.g. tattoo ink, cancers, and PDT drugs), hair follicles, hair bulge cells, and vascular tissue. Example absorption spectra that can be used to distinguish human fatty tissue from water based tissue are given in FIGS. 1 and 2 for a range of optical wavelengths.

Alternatively, a cheaper sensing element 170 can be implemented by measuring absorption or scattering properties using a broadband source with a single photodetector to measure absorption without the need for a spectral filter. However the sensitivity of such a sensing element would be dramatically reduced in comparison to a multiwavelength sensor. A narrow wavelength illumination source (e.g., a laser or LED), could be used with a photodetector to produce a low cost sensor that would allow the optimization of the chosen wavelength to create maximum distinction between the lipid-rich target and the surrounding tissue and thus improve the sensitivity of the sensor relative to a comparable sensor that is combined with a broad band source.

The sensing element 170 can alternatively be an acoustic transducer. An acoustic transducer can be used, for example, to measure a signal generated as the result of ablation of skin 190. For example, an acoustic transducer could detect a characteristic (e.g., magnitude, frequency, resonance, or time of flight) of the small popping sound associated with the sudden expansion of tissue due to laser ablation. Since tissue material properties such as elasticity, absorption, and refractive index may affect the popping sound characteristics, the characteristics of the popping sound may correspond to the type of material being ablated and thus may be used to distinguish types of material such as lipid-rich material. This type of sensor has the advantage of being able to detect signals by nonoptical means, which reduces the need to clean sensitive optical components. It also has the advantage of allowing the signatures of lipid-rich targets lying in the region just below the hole by measuring changes in the signal resonance of one or more acoustical transducers. Multiple transducers may be used to more precisely locate (e.g., through triangulation) or to determine the extent of particular lipid-rich targets.

The sensing element 170 can be an effluent detector that detects the volume of ablated material or a rate of ablation. An effluent detector can be implemented using the optical absorption properties of a broadband source on a broad area detector to measure the approximate volume of material that is ejected during ablation. An effluent detector can also be a piezoresistive element that changes resistivity or a resonant crystal that changes resonance characteristics in response to small changes in the amount of incident ablation material. These types of detectors can be very accurate for determining the ablation rate. Care must be taken during design to prevent the detectors from becoming overloaded during treatment, which can reduce sensitivity.

The sensing element 170 can be a strobe light and a CCD camera that captures images of ablated material to measure the trajectory, velocity, or amount of ablated material that is ejected from the skin.

The sensing element 170 can also comprise a combination of elements, such as the combination of an acoustic sensor and a spectral sensor. A combination sensor would improve the reliability of the sensing element 170 and would allow for more complex functionality to be integrated into the system.

The electromagnetic source 110 ablates the skin 190 to create multiple holes. The electromagnetic source 110 can be chosen based on the desired treatment characteristics. The electromagnetic source 110 can be an optical source, an RF source, an RF plasma source, or a combination thereof. The electromagnetic source 110 can be chosen based on the electrical driver requirements, power, cost, size, and reliability. Properties of the emitted electromagnetic energy 115 should also be considered such as how the energy 115 will be scattered and absorbed by the tissue. For example, it may be desired to limit the maximum diameter of the holes, in which case, a electromagnetic source 110 that is highly absorbing and can be tightly focused could be distinguishing features in selecting the electromagnetic source 110, for example an Er:YAG laser. A less highly absorbing electromagnetic source 110, such as a CO₂ laser, may be desired in order to create a thermal coagulation zone surrounding the perimeter of the hole during ablation, which can beneficially cause tissue shrinkage and reduce bleeding in comparison to more strongly ablative choices. In embodiments where optical sources are used, electromagnetic sources 110 with infrared wavelengths are preferred over visible and ultraviolet wavelengths in applications where optical scattering is important, for example in nonablative treatment of a deep target with a small beam size, because scattering is lower in the infrared wavelengths.

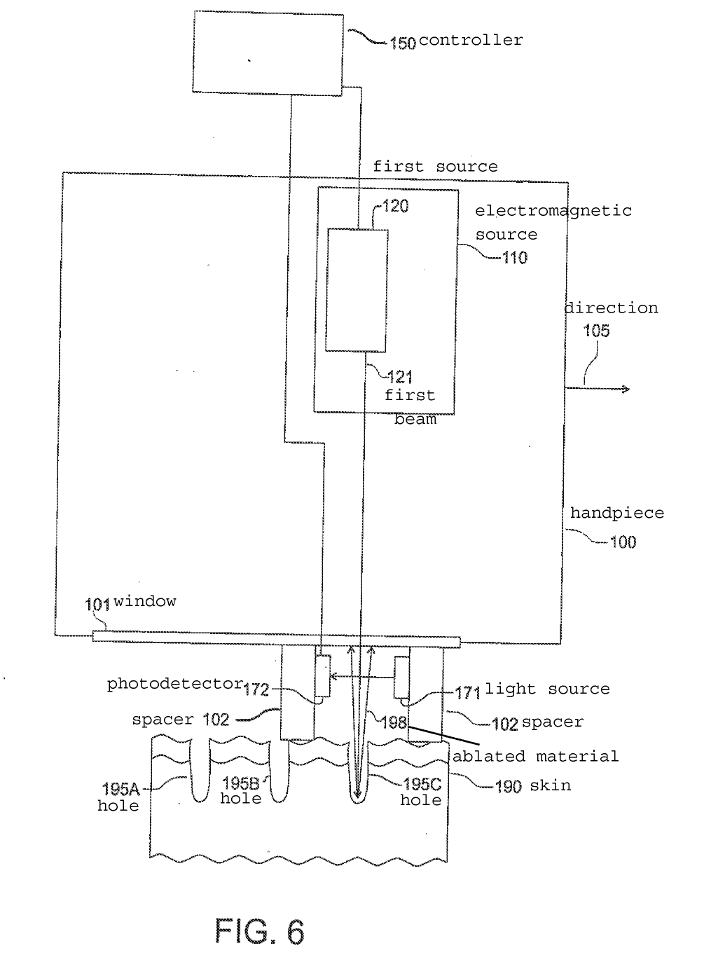

The electromagnetic source 110 may beneficially combine multiple energy sources to draw on the characteristic features of different types of sources. For example, as shown in FIG. 5, the electromagnetic source 110 can comprise a first source 120 and a second source 130. The first source 120 may be selected for optimal characteristics for the ablative component of the treatment while the second source 130 can be selected for characteristics that would be optimized for nonablative treatment. Ablative sources, such as a CO₂ laser with a wavelength of approximately 10.6 μm, an Er:YAG laser with a wavelength of approximately 2.94 μm, a Holmium laser with a wavelength of approximately 2.14 μm, a Thulium-doped fiber laser with a wavelength of approximately 1.92 μm (e.g., model TLR-50-1920 from IPG Photonics, Inc., Oxford, Mass.) or with a wavelength in the range of 1870-2100 nm where the absorption in tissue is high enough to create ablation with a tightly focused beam, a RF plasma system, or a combination thereof, can be combined with nonablative sources to create the electromagnetic source 110. Examples of second sources that can be used for nonablative treatment include diode lasers, RF sources, RF plasma sources, erbium fiber lasers, diode lasers amplified by erbium-doped fiber amplifiers, optical parametric amplifiers (OPAs), or other optical amplifiers, ytterbium-doped fiber lasers, thulium-doped fiber lasers, Nd:YAG lasers, Raman-shifted fiber lasers, optical parametric oscillators (OPOs), and dye lasers.

The first source 120 and second source 130 that are combined in FIG. 5 are optical sources. Other combinations and appropriate system modifications can be easily visualized by those skilled in the art without the need for additional figures. The electromagnetic source 110 could comprise, for example, one or more of the set of above mentioned ablative sources with one or more of the set of above mentioned nonablative sources. The choice of a particular ablative source can be made based on the degree of coagulation that is desired during the ablation step, the desire for fiber delivery to the handpiece, the desired hole depth and diameter, and the cost sensitivity for the laser system. The choice of a particular nonablative second source can be made based on the desired thermal heat profile, the absorption characteristics of the target to be heated, the absorption characteristics of surrounding tissue, the desired beam size, and the cost sensitivity of the laser system.

In some embodiments, holes are ablated with a laser having a water absorbed wavelength (i.e. a wavelength that has a higher absorption coefficient in water than in human fatty tissue) and the at least one pulse of electromagnetic energy is produced by a laser having a fat selective wavelength (i.e. a wavelength that has a higher absorption coefficient in human fatty tissue than in water). The use of an ablative water absorbing wavelength has the advantage of being less selective as tissue is ablated. The use of a fat selective wavelength for the at least one pulse of electromagnetic energy has the advantage of preferentially targeting lipid-rich targets in comparison to the surrounding tissue and thus reducing side effects by reducing collateral damage surrounding the desired target. Thus, the combined use of a water absorbed wavelength and a fat selective wavelength can provide non-selective ablation to a desired depth and selective treatment of a selected target. For example, a CO₂ laser can be used with a ytterbium-doped fiber laser that is Raman shifted, preferably to emit a peak wavelength in the range of about 1.19-1.22 μm, or with an erbium-doped fiber laser that is Raman shifted, preferably to emit a peak wavelength in the range of about 1.69-1.73 μm. The particular uses of these lasers provide good selectivity for fat over water and limited water absorption in tissue to reduce collateral damage. Both of these lasers have the additional advantage of being lower cost than sources such as OPOs or free electron lasers that are less desirable for commercial deployment in cost sensitive applications. The Raman shifted erbium-doped fiber laser will advantageously be more selective in fat and substantially more absorbing in fat than the Raman shifted erbium-doped fiber laser but will also be more expensive.

In some embodiments, holes are ablated with a laser having a water absorbed wavelength and the at least one pulse of electromagnetic energy is produced by a laser having a water absorbed wavelength. The advantage of using a water absorbing wavelength for the nonablative treatment pulse is that more uniform thermal profiles can be created throughout a target that is reached through ablation. In a particular embodiment, a CO₂ laser is combined with an erbium doped fiber laser emitting in the range of about 1.50-1.65 μm, or more preferably in the range of 1.53-1.60 μm. An erbium doped fiber laser in this wavelength range has the advantage that it can be matched to the approximate size of the target to create an optimal deposition of treatment energy throughout the region that contains the target. Er:glass lasers, InGaAs based laser diode arrays, and laser diodes amplified by erbium-doped fiber amplifiers can be used in place of the erbium-doped fiber laser.

As shown in FIG. 6, the electromagnetic source 110 can alternatively include exactly one optical source. In a preferred embodiment, holes can be drilled into the skin 190 where the electromagnetic energy 115 is more strongly absorbed by water than by lipid-rich tissue. For example, the electromagnetic energy 115 could be optical energy that is emitted, for example, from an electromagnetic source 110 that comprises a CO₂ laser, an Er:YAG laser, a Holmium laser, or a Thulium-doped fiber laser. With the appropriate choice of wavelength, pulse energy, pulse power, focal depth, surface cooling, and spot size, the electromagnetic energy 115 can be ablative in tissue that is comprised predominantly of water, for example in dermal tissue which is typically 60-80% water, and nonablative in tissue that is lipid-rich, for example in sebaceous glands or subcutaneous fat. For example, the absorption of 1.92 μm wavelength light emitted from a thulium-doped fiber laser has an absorption coefficent of approximately 90 cm⁻¹ in tissue containing 70% water and can have an absorption coefficient as low as approximately 2 cm⁻¹ in lipid-rich tissue. This can be beneficially used to deposit heat to drill down to a sebaceous gland using a small hole of less than 1 mm in diameter and then nonablatively deposit heat in the sebaceous gland that may be larger than 1 mm in diameter without changing the treatment parameters. Thus, the treatment effects can be similar to those accomplished by delivering two separate sets of parameters for the electromagnetic energy 115 during an ablation step and a nonablative treatment step, as illustrated in FIG. 4C, without incorporating two separate sources.

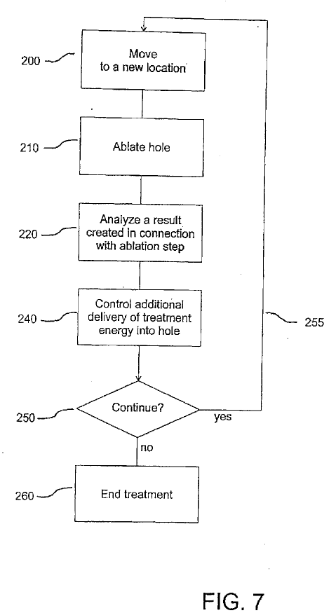

A method for using the inventive apparatus is described in FIG. 7. The method comprises the steps of moving 200 handpiece 100 to a new location, ablating 210 at least one hole, analyzing 220 a result created in connection with the ablating step 210, controlling 240 the delivery of electromagnetic energy 115 into the hole created during the ablating step 210 based on the result of the analyzing step 220, deciding 250 whether to continue treatment, and ending 260 treatment. In the inventive method, the decision path 255 indicated by continuing to the method is followed at least once to form a pattern of at least two ablated holes that are created during the ablating step 210. The analyzing step 220 uses a sensing element 170.

FIG. 5 shows an embodiment of the invention wherein the electromagetic source 110 comprises a first source 120, a second source 130, a mirror 141, and a dichroic mirror 142. The mirror 141 reflects the first beam 121 from the first source 120 to the dichroic mirror 142, which combines the first beam 121 with a second beam 131 from the second source into a combined beam 135. The combined beam 135 is received by an embodiment of the delivery system that comprises a receiving mirror 143 that deflects the combined beam 135 into an optical scanner 145, examples of which were described above. In a preferred embodiment, the optical scanner 145 is a starburst scanner. The scanner deflects the combined beam 135 to one or more locations on the skin 190 to ablate tissue, thus creating a plume of ablated material 198. The ablated material 198 can be detected by the photodetector 172 when illuminated by the light source 171. The ablation event may also generate an acoustical signal that is detected by an ultrasonic transducer 173. An optical mouse sensor 161 is used to measure the velocity of the handpiece 100 as the handpiece moves across the skin 190 along direction 105. The first source 120 and second source 130 are controlled by the controller 150. The electromagnetic energy 115 is delivered through a transparent handpiece window 101, which seals the optical scanner 145 from the ablated material 198. Spacers 102 are used to maintain a desired distance between the optical scanner 145 and the skin 190 so that the skin 190 is in the desired focal position of the combined beam 135.

Note that the combined beam may not include the first beam 121 and the second beam 131 at the same time. The term combined beam 135 simply provides a shorthand notation for describing the one or more beams that is being received by delivery system 140 from the electromagnetic source 110.

Although the detailed description contains many specifics, these should not be construed as limiting the scope of the invention but merely as illustrating different examples and aspects of the invention. It should be appreciated that the scope of the invention includes other embodiments not discussed in detail above. For example, the system may optionally include vacuum suction or pressured airflow to remove ablative effluent. The system may optionally also provide cooling to reduce pain and to spare epidermal tissue to reduce side effects. Any of the described embodiments for the electromagnetic source 110 can be combined with any of the described embodiments for the sensing elements 170 and optionally with any of the described embodiments for the positional sensor to produce an apparatus and method according to the invention. The advantages of such combinations will be clear to those skilled in the art. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

Without limiting the scope of the above disclosure, each aspect of the inventive method is further designed to be directed to a method of cosmetic dermatological treatment, and more specifically to a method of non-invasive cosmetic dermatolgical treatment.

The terms tissue and skin are used interchangeably in this application to refer to in vivo human skin.

In the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

1. A method of dermatological treatment comprising the steps of
ablating epidermal and dermal tissue, in a dermatological treatment, to form discrete holes in a selected region of skin, the ablation step resulting in both ablated tissue and remaining tissue that was not ablated within the selected region;
evaluating, with a sensing element, at least a portion of tissue from the selected region in connection with the ablating step for a selected tissue target;
detecting, in response to the evaluation, the selected tissue target in tissue surrounding a base of at least one of the discrete holes;
gathering, in response to the detection, at least one energy-delivery parameter of an electromagnetic source to begin emission of nonablative electromagnetic energy; and
applying at least one pulse of the nonablative electromagnetic energy into an inside of the hole at which the selected tissue target was detected for treating the selected tissue target with the nonablative electromagnetic energy.

2. (canceled)

3. A method of claim 1, wherein the detecting step comprises detecting a presence of at least one of hair follicles, hair bulge cells, and vascular tissue.

4. A method of claim 1, wherein the detecting step comprises detecting a presence of lipid-rich tissue.

5. (canceled)

6. A method of claim 4, wherein the evaluating step comprises measuring a characteristic of a portion of tissue that contains at least part of the ablated tissue and wherein the measured characteristic comprises at least one of an ablation rate, a fluorescent emission of the portion of tissue, a scattering property or an absorption property of the portion of tissue for at least one optical wavelength, and an optical absorption or scattering of the portion of tissue at at least two wavelengths.

7-10. (canceled)

11. A method of claim 4, wherein the evaluating step comprises measuring a characteristic of a portion of tissue that contains at least part of the remaining tissue underlying at least one of the holes, the measured characteristic comprising a characteristic selected from the group consisting of: an acoustical or radio-frequency absorption spectrum of the portion of tissue, a fluorescent emission of the portion of tissue, a depth of at least one of the holes, a scattering property and an absorption property of the portion of tissue for at least one optical wavelength, and an optical absorption of the portion of tissue at at least two wavelengths

12-21. (canceled)

22. A method of claim 4, wherein the applying step comprises creating a nonablative thermal treatment zone at the base of the hole where the lipid-rich tissue was detected.

23. (canceled)

24. A method of claim 4, wherein the at least one pulse of nonablative electromagnetic energy is emitted from an optical source and the optical source emits energy at an infrared fat selective wavelength or an infrared water absorbed wavelength.

25. (canceled)

26. A method of claim 4, wherein the ablating step comprises the step of directing a laser beam to the selected region to heat water in the selected region, and wherein at least two of the discrete holes are created in a pattern corresponding to the optical intensity profile of the laser beam.

27-28. (canceled)

29. A method of claim 26, wherein the at least one pulse of nonablative electromagnetic energy is emitted from a second laser, and wherein the altering step further comprises the step of delivering a beam from an optical source comprising at least one of the laser and the second laser to at least two of the holes to cause treatment of at least one lipid rich target.

30-31. (canceled)

32. A method of claim 26, wherein the laser comprises a CO₂ laser and the at least one pulse of nonablative electromagnetic energy is emitted from a Raman-shifted fiber laser or at least one of an erbium-doped fiber laser and an erbium-doped fiber amplifier.

33-34. (canceled)

35. A method of claim 4, further comprising the step of
applying an absorbing agent applied to the surface of the selected region and
wherein the ablating step comprises the step of directing a laser beam to the absorbing agent.

36. A method of claim 4, wherein the density of holes is 100-10,000 per square centimeter in the selected region.

37. A method of claim 36, wherein the density of holes is 1000-2000 per square centimeter in the selected region.

38. A method of claim 4, wherein the altering step consists of the step of activating the delivery of the at least one pulse of nonablative electromagnetic energy in response to the result of the evaluating step.

39. A method of claim 4, wherein the altering step comprises the step of selecting at least one location at the selected region for delivery of the at least one pulse of nonablative electromagnetic energy in response to the result of the evaluating step.

40. (canceled)

41. A method of claim 4, further comprising focusing the at least one pulse of nonablative electromagnetic energy using an optical lens array.

42. A method of claim 4, wherein at least one of the holes has a depth of 0.5-6 mm and a diameter of 0.2-2.0 mm.

43. An apparatus for dermatological treatment comprising:
an electromagnetic source configured to emit ablative and nonablative electromagnetic energy;
a delivery system configured to deliver the ablative electromagnetic energy to multiple discrete locations at a selected region of skin to form a pattern of discrete holes in the selected region including both ablated tissue and remaining tissue that was not ablated within the selected region;
a sensing element configured to evaluate at least a portion of the tissue from the selected region for a selected tissue target and detect the selected tissue target in tissue surrounding a base of at least one of the discrete holes;
a controller configured to, in response to detection data received from the sensing element, alter at least one energy-delivery parameter of the electromagnetic source to begin emission of nonablative electromagnetic energy; and
the delivery system further configured to apply at least one pulse of the nonablative electromagnetic energy into an inside of the hole at which the selected tissue target was detected for treating the selected tissue target with the nonablative electromagnetic energy.

44. An apparatus of claim 43, wherein the electromagnetic source includes exactly one laser or at least two lasers.

45. (canceled)

46. An apparatus of claim 43, wherein the electromagnetic source comprises at least two optical sources with different optical emission spectra.

47. An apparatus of claim 43, wherein the electromagnetic source comprises at least one of a CO₂ laser, a thulium-doped fiber laser that is configured to be tunable, an Er:YAG laser, a Raman-shifted fiber laser, an erbium-doped fiber laser with an erbium-doped fiber amplifier, a fiber laser, a CO₂ laser with at least one of a flashlamp or a radio-frequency source, and a holmium laser.

48-53. (canceled)

54. An apparatus of claim 43, wherein the electromagnetic source emits energy at an infrared fat-selective wavelength or an infrared water-selective wavelength.

55. (canceled)

56. An apparatus of claim 43, wherein the sensing element is configured to measure a characteristic of the portion of tissue only after the portion of tissue has been ablated, the measured characteristic selected from the group consisting of: an ablation rate, a scattering property or an absorption property of the portion of tissue for at least one optical wavelength a fluorescent emission of the portion of tissue and an optical absorption of the portion of tissue for least two wavelengths.

57-60. (canceled)

61. An apparatus of claim 43, wherein the sensing element comprises an element selected from a group consisting of: an ultrasonic transducer, an optical source with an optical detector, and a spectral filter.

62-63. (canceled)

64. An apparatus of claim 43, wherein the electromagnetic source comprises an optical source that emits light with a wavelength of 350-450 nm.

65. An apparatus of claim 43, wherein the electromagnetic source is further configured to create a nonablative thermal treatment zone at the base of the hole where the selected tissue target was detected.

66. An apparatus of claim 43, further comprising a controller that independently controls parameters of the ablative and nonablative electromagnetic energy that affect the dermatological treatment.

67. An apparatus of claim 66, further comprising a positional sensor that measures at least one of the relative position, relative velocity, relative speed, and relative acceleration between the handpiece and the selected region.

68. An apparatus of claim 67, wherein the controller is further configured to receive data from the positional sensor and control at least one parameter of the electromagnetic source that affects dermatological treatment in response to data received from the positional sensor.

69. An apparatus of claim 43, wherein the delivery system comprises a system selected from the group consisting of: an optical scanner, an optical lens array, and a patterned mask.

70-71. (canceled)
